(19) United States
(12) Patent Application Publication
(10) Pub. No.: US 2025/0367007 A1
(43) Pub. Date: Dec. 4, 2025

(54) SIDE ENTRY GLENOSPHERE INSERTION DEVICE AND METHOD OF USE

(71) Applicants: Nicholas J. Schoch, Shelby Township, MI (US); Paul A. Zwirkoski, Pinckney, MI (US)

(72) Inventors: Nicholas J. Schoch, Shelby Township, MI (US); Paul A. Zwirkoski, Pinckney, MI (US)

(21) Appl. No.: 19/224,668

(22) Filed: May 30, 2025

Related U.S. Application Data
(60) Provisional application No. 63/731,700, filed on May 30, 2024.

(51) Int. Cl. A61F 2/46; A61F 2/40

(52) U.S. Cl. CPC A61F 2/4612; A61F 2/4081; A61F 2002/4622

(57) ABSTRACT

The present disclosure relates in general to surgical instruments and methods of use. More specifically, but not exclusively, the present disclosure relates to an insertion device and a method for use for reverse shoulder replacement surgery. The surgical insertion device of the present disclosure comprises a handle portion, an armature portion, and a tip portion that are designed for side entry glenosphere insertion. In some instances, the surgical insertion device comprises an adjustable arm/tip orientation. In other instances, the surgical insertion device comprises a fixed curved tip orientation. In both instances, the surgical insertion device is designed to improve access to surgical sites while mitigating surrounding tissue and/or nerve damage.

### CROSS-REFERENCE TO RELATED APPLICATIONS

This application claims priority under 35 U.S.C. § 119 to U.S. Provisional Patent Application No. 63/731,700 filed May 30, 2024, herein incorporated by reference in its entirety.

### TECHNICAL FIELD

The present disclosure relates in general to surgical instruments and methods of use. More particularly, the present disclosure relates to an insertion device and a method for use for reverse shoulder replacement surgery. The purpose of the present disclosure is to provide an insertion device that is capable of side entry glenosphere insertion for reverse shoulder replacement surgery.

### BACKGROUND

Orthopedic surgeries often provide limited access to the surgical site that is complicated by the risk of damaging surrounding tissue and nerves during surgery. Surgical insertion devices traditionally utilized in the medical industry for reverse shoulder replacement surgery typically provide a fixed arm/tip orientation that are configured for straight-on glenosphere insertion involving a direct anterior incision (e.g., deltopectoral approach). Complicating matters with such traditional insertion devices for reverse shoulder replacement surgery is the position of the humerus which creates an obstacle during insertion of the glenosphere. Because of their fixed arm/tip orientation, traditional surgical insertion devices also fail to provide adjustability for optimal placement and control of the glenosphere during reverse shoulder replacement surgery. Moreover because the structure of these traditional insertion devices are highly specialized, such devices also cannot be universally applied across all sizes of glenospheres.

As a result, a need exists for an improved surgical insertion device and method of use that provide for side entry insertion of the glenosphere during reverse shoulder replacement surgery that avoids obstacles such as the humerus. A need further exists for an improved surgical insertion device and method of use that provide an adjustable arm/tip orientation for optimal placement and control of the glenosphere during reverse shoulder replacement surgery. A need further exists for an improved surgical insertion device and method of use that may be universally applied across all sizes of glenospheres.

### SUMMARY

In one embodiment of the present disclosure, a surgical insertion device is provided. The surgical insertion device may comprise a handle portion, an armature portion, and a tip portion. The surgical insertion device may be designed for side entry glenosphere insertion for reverse shoulder replacement surgery.

In another embodiment of the present disclosure, a surgical insertion device is provided. The surgical insertion device may comprise a handle portion having at least one handle, a pivot connection joint, and at least one shank connecting the at least one handle to the pivot connection joint. The surgical insertion device may further comprise an armature portion having at least one arm, each arm having a proximal end and a distal end, the proximal end of each arm connected to the pivot connection joint of the handle portion, and at least one suture tether configured to removably attach the distal end of each arm of the armature portion to the tip portion. The surgical insertion device may also comprise a tip portion having at least one locking assembly, at least one variable angled tip, and the distal end of the arm of the armature portion configured to removably attach to the locking assembly of the tip portion through tensioning the suture tether. The surgical insertion device may be designed to provide adjustable arm/tip orientation.

In yet another embodiment of the present disclosure, a method of using a surgical insertion device is provided. The method may comprise providing a surgical insertion device having a handle portion, an armature portion, and a tip portion. In particular, the tip portion may comprise at least one of a variable angled tip or a fixed curved tip. The surgical insertion device may be used to perform side entry glenosphere insertion for reverse shoulder replacement surgery.

### OBJECTS, FEATURES, AND ADVANTAGES

It is a principal object, feature, and advantage of the present disclosure to overcome the aforementioned deficiencies in the art and provide an improved surgical insertion device and method of use that provide for side entry insertion of the glenosphere during reverse shoulder replacement surgery that avoids obstacles such as the humerus.

Another object, feature, and advantage of the present disclosure is to provide an improved surgical insertion device and method of use that provide an adjustable arm/tip orientation for optimal placement and control of the glenosphere during reverse shoulder replacement surgery.

Yet another object, feature, and advantage of the present disclosure is to provide an improved surgical insertion device and method of use that may be universally applied across all sizes of glenospheres.

A further object, feature, and advantage of the present disclosure is to provide an improved surgical insertion device and method of use that provide three point fixation on the glenosphere for better control, maneuverability, and insertion of the implant during reverse shoulder replacement surgery.

A still further object, feature, and advantage of the present disclosure is to provide an improved surgical insertion device and method of use that mitigate risk of damage to surrounding tissue and nerves during reverse shoulder replacement surgery.

Another object, feature, and advantage of the present disclosure is to provide an improved surgical insertion device and method of use that is cost efficient to produce while maintaining structural integrity for long term use.

Other objects, features, or advantages of this disclosure will become apparent from the following detailed description and claims, taken in conjunction with the accompanying drawings that set forth, by way of illustration and example and without limitation, certain aspects of this disclosure. No single aspect need provide each and every object, feature, or advantage. Thus the present disclosure is not to be limited to or by these objects, features, and advantages.

### BRIEF DESCRIPTION OF THE DRAWINGS

The accompanying drawings, incorporated herein and forming a part of the specification, illustrate aspects of the present disclosure together with the detailed description and claims.

FIG. 1 is an isometric view of a traditional surgical insertion device with a fixed arm/tip configuration (prior art).

FIG. 2 is an isometric view of an alternative embodiment of the traditional surgical insertion device with a fixed arm/tip configuration (prior art).

FIG. 3 is an isometric view of a first embodiment of the surgical insertion device of the present disclosure.

FIG. 4 is a detail top view of the surgical insertion device of FIG. 3, the bottom side view being a mirror image thereof.

FIG. 5 is a detail right side view of the surgical insertion device of FIG. 3, the left side view being a mirror image thereof.

FIG. 6 is a detail top view of the surgical insertion device of FIG. 3, the bottom side view being a mirror image thereof.

FIG. 7 is a detail right side view of the surgical insertion device of FIG. 3, the left side view being a mirror image thereof.

FIG. 8 is a detail right side cross-sectional view of the surgical insertion device of FIG. 3.

FIG. 9 is a detail top rear isometric exploded view of the surgical insertion device of FIG. 3.

FIG. 10 is a detail right side cross-sectional view of the surgical insertion device of FIG. 3, further disclosing nonlimiting examples of dimension ranges.

FIG. 11 is a top view of a second embodiment of the surgical insertion device of the present disclosure, further disclosing nonlimiting examples of dimension ranges.

FIG. 11A is a left side view of the surgical insertion device of FIG. 11, the right side view being a mirror image thereof.

FIG. 11B is a cross-sectional view A-A of the surgical insertion device of FIG. 11.

FIG. 12 is a top view of another aspect of the surgical insertion device of FIG. 11, further disclosing nonlimiting examples of dimension ranges.

FIG. 12A is a left side view of the surgical insertion device of FIG. 12, the right side view being a mirror image thereof.

FIG. 12B is a cross-sectional view B-B of the surgical insertion device of FIG. 12.

FIG. 13 is a top front isometric view of another aspect of the surgical insertion device of FIG. 11, further disclosing nonlimiting examples of dimension ranges.

FIG. 13A is a cross-sectional view D-D of the surgical insertion device of FIG. 13.

FIG. 13B is a cross-sectional enlarged view E of the surgical insertion device of FIG. 13.

FIG. 14 is a top front isometric view of another aspect of the surgical insertion device of FIG. 11, further disclosing nonlimiting examples of dimension ranges.

FIG. 14A is a cross-sectional view F-F of the surgical insertion device of FIG. 14.

FIG. 14B is a cross-sectional enlarged view H of the surgical insertion device of FIG. 14.

FIG. 15 is a top view of another aspect of the surgical insertion device of FIG. 11, the bottom view being a mirror image thereof, further disclosing nonlimiting examples of dimension ranges.

FIG. 15A is a right side view of the surgical insertion device of FIG. 15, the left side view being a mirror image thereof.

FIG. 15B is a detail bottom view of the surgical insertion device of FIG. 15.

FIG. 16 is a top view of another aspect of the surgical insertion device of FIG. 11, the bottom view being a mirror image thereof, further disclosing nonlimiting examples of dimension ranges.

FIG. 16A is a right side view of the surgical insertion device of FIG. 16, the left side view being a mirror image thereof.

FIG. 16B is a detail bottom view of the surgical insertion device of FIG. 16.

FIG. 17 is a top left side view of a third embodiment of the surgical insertion device of the present disclosure.

FIG. 18 is a bottom right side view of the surgical insertion device of FIG. 17.

### DETAILED DESCRIPTION

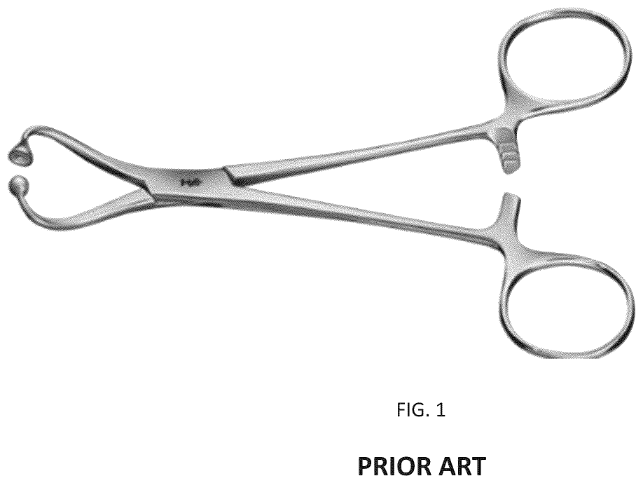

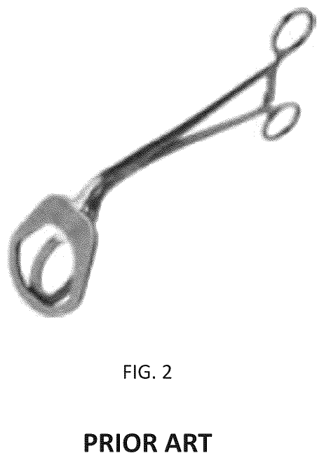

Illustrated in FIGS. 1-2, traditional surgical insertion devices for reverse shoulder replacement surgery typically comprise a fixed arm/tip orientation that is configured for straight-on glenosphere insertion. In particular, FIG. 1 shows an example of a pinched fixed arm/tip geometry. FIG. 2 shows an example of a fixed angled arm having a custom tip. As explained above, such traditional surgical insertion devices fail to provide for side entry insertion of the glenosphere that avoids obstacles such as the humerus. Such traditional surgical insertion devices also cannot be used with all sizes of glenospheres. Nor do these traditional surgical insertion devices provide three point fixation on the glenosphere. These traditional surgical insertion devices further lack an adjustable arm/tip orientation for optimal placement and control of the glenosphere during reverse shoulder replacement surgery.

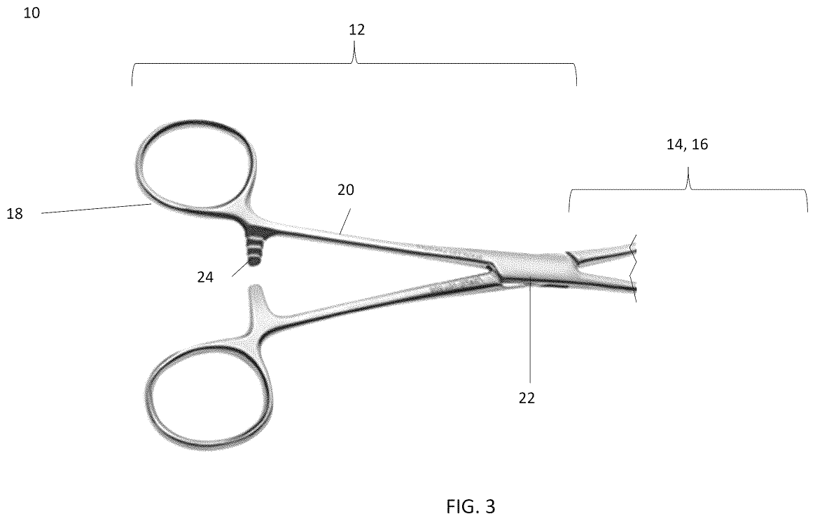

Referring generally to FIGS. 3-18, the present disclosure relates to surgical insertion devices and methods that provide for side entry insertion of the glenosphere during reverse shoulder replacement surgery that avoids obstacles such as the humerus. The surgical insertion devices and methods of the present disclosure may include an adjustable arm/tip orientation for optimal placement and control of the glenosphere during reverse shoulder replacement surgery. The surgical insertion device and methods of the present disclosure are designed to be universally applicable across all sizes of glenospheres. The surgical insertion devices and methods of the present disclosure may also provide three point fixation on the glenosphere for better control, maneuverability, and insertion of the implant during reverse shoulder replacement surgery. The surgical insertion devices and methods of the present disclosure are further designed to mitigate risk of damage to surrounding tissue and nerves during reverse shoulder replacement surgery. While intended for human applications, nevertheless, the surgical insertion devices and methods of the present disclosure may also be utilized in large or small animal veterinary applications.

While certain aspects of the present disclosure are shown and described herein, it is understood that such aspects are merely exemplary. The present disclosure is not intended to be limited to these specific aspects and may encompass other aspects or embodiments. Therefore, specific system and method details disclosed herein are not to be interpreted or inferred as limiting, but merely as a basis for the claims and as a representative basis for teaching one skilled in the art how to make and use the disclosed subject matter.

It must further be noted that the singular terms “a,” “an,” and “the” as used herein may include plural referents unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means.

All words of approximation as used in the present disclosure and claims should be construed to mean “approximate,” rather than “perfect” or “exact,” and may be used as a modifier to any other word, number, quantity, quality, value, or specified parameter. Words of approximation, include, but are not limited to terms such as “about,” “approximately,” “around,” “almost,” “generally,” “largely,” “essentially,” “substantially,” etc. As used herein, in some aspects, the terms “about” or “approximately” when preceding a numerical value may indicate the value plus or minus a range of 0.001, 0.002, 0.003, 0.004 or 0.005 inches. In other aspects, the terms “about” or “approximately” when preceding a numerical value may indicate the value plus or minus a range of 0.01, 0.02, 0.03, 0.04 or 0.05 inches. In further aspects, the terms “about” or “approximately” when preceding a numerical value may indicate the value plus or minus a range of 1, 2, 3, 4 or 5 millimeters. In still further aspects, the terms “about” or “approximately” when preceding a numerical value may indicate the value plus or minus a range of 1, 2, 3, 4 or 5 degrees. In some aspects, the terms “about” or “approximately” when preceding a numerical value may indicate the value plus or minus a range of 0.001, 0.002, 0.003, 0.004 or 0.005 radians/inches (R # inches). In other aspects, the terms “about” or “approximately” when preceding a numerical value may indicate the value plus or minus a range of R.01, R.02, R.03, R.04 or R.05 inches.

Furthermore the transitional phrase “comprising” that is synonymous with “including,” “containing,” and “characterized by” as used herein is inclusive or open-ended and does not exclude additional, unrecited elements, steps or ingredients. Alternatively the transitional phrase “consisting of” as used herein is closed and excludes any element, step or ingredient not specified. The transitional phrase “consisting essentially of” limits the scope of a claim to the specified materials or steps and those that do not materially affect the basic and novel characteristics of the claims.

Illustrated in FIGS. 3-14, the surgical insertion device (10) may comprise different aspects that vary in size, thickness, and intended use. In some instances, the surgical insertion device (10) may comprise an overall length ranging between approximately 6-30 inches. In other instances, the surgical insertion device (10) may comprise an overall length ranging between approximately 8-12 inches. In further instances, the surgical insertion device (10) may comprise an overall thickness ranging between approximately 0.125-0.750 inches. The foregoing dimension ranges are nonlimiting, as the overall length and thickness of the surgical insertion device (10) may be optimized for effective use during reverse shoulder replacement surgery. The surgical insertion device (10) and/or components thereof may be comprised of medical grade stainless steel noncorrosive materials, medical grade polymers, or combinations thereof which are provided sterile and intended for repeat applications after appropriate sterilization reprocessing. Alternatively, the surgical insertion device (10) and/or components thereof may be comprised of medical grade stainless steel noncorrosive materials, medical grade polymers, or combinations thereof which are provided sterile and intended for single use applications that are disposable and/or recyclable.

Illustrated in FIGS. 3-10, a first embodiment of the surgical insertion device (10) may include three primary regions, namely, a handle portion (12), an armature portion (14), and a tip portion (16). In particular, the handle portion (12) may comprise at least one handle (18) designed to allow a surgeon to reliably grip the instrument during surgical procedures (i.e., using hand, fingers, and/or palm). Each handle (18) may be connected to a shank (20), wherein the shanks (20) converge together at a pivot connection joint (22) located approximately midpoint of the surgical insertion device (10). The pivot connection joint (22) may be configured to rotatably couple the shanks (20) while providing approximately 45°-360° rotation. In some instances, the handle portion (12) may include an optional fastening mechanism (24), such as, but not limited to, a ratchet or box lock. The fastening mechanism (24) may be configured to allow the surgical insertion device (10) to clamp onto tissue or an implant securely and therefore reduce a surgeon's hand fatigue during prolonged surgical procedures.

Shown in FIGS. 3-10, the armature portion (14) of the surgical insertion device (10) may comprise at least one arm (26). Each arm (26) may comprise a proximal end (28) connected to the pivot connection joint (22) and an opposite distal end (30). In some instances, the distal end (30) of each arm (26) is configured for removable attachment to the tip portion (16) through at least one suture tether (32). In such instances, the at least one suture tether (32) may be comprised of monofilament or multifilament polymer yarns that are biodegradable and for single-use application. In other instances, the at least one suture tether (32) may be comprised of monofilament or multifilament polymer yarns that are reusable after appropriate sterilization procedures. In further instances, the handle portion (12) and the armature portion (14) may be provided together as a variable pivoting instrument kit (“V-PIK”). The V-PIK may be provided as a re-usable portion of the final assembly of the surgical insertion device (10) reusable after appropriate sterilization procedures. Alternatively, the V-PIK may be provided as a disposable and/or recyclable, one-use portion of the final assembly of the surgical insertion device (10).

Further shown in FIGS. 3-10, the tip portion (16) of the surgical insertion device (10) may comprise at least one locking assembly (34) and at least one variable angle tip (36). In particular, the locking assembly (34) of the tip portion (16) may be removably connected to the distal end (30) of the arm (26) using the suture tether (32). In some instances, the suture tether (32) may be configured to be pulled toward the distal end (30) of the arm (26) where the locking assembly (34) locks into position as confirmed with an audible “click” sound. The at least one variable angle tip (36) may be configured to provide angular adjustment over six degrees of freedom (“DOF”), namely, the x axis, y axis, and z axis, with respect to the at least one locking assembly (34). The at least one variable angle tip (36) may be further configured to provide rotational adjustment about the x axis, y axis, and z axis, with respect to the at least one locking assembly (34). Such adjustability of the at least one variable angle tip (36) provides access to the front of the glenosphere for better accessibility to the base plate during trialing and implantation.

Still further shown in FIGS. 3-10, the particular designs of the at least one variable angle tip (36) may include rectangular captures, circular captures, forked captures, rake-like retraction captures, spike-like piercing control captures, and shovel-like leveraging captures. In some instances, the particular designs of the at least one variable angle tip (36) may include fixed, modular, or variable designs, and combinations thereof. In other instances, the particular designs of the at least one variable angle tip (36) may provide for intra-operative adjustments during surgical interventions wherein the surgeon may adjust each variable angle tip (36) independently, and then set and lock the particular tip orientation with respect to its respective locking assembly (34). In further instances, the particular designs of the at least one variable angle tip (36) may provide for dis-engagement with the locking assembly (34) for disassembly and/or retrieval of the surgical insertion device (10) via the suture tethers (32). Therefore, the at least one variable angle tip (36) is configured to provide for intra-operative adjustability in orientation over six DOF, while further providing for the capability to lock and unlock the variable angle tips (36) in position during surgical intervention, in addition to dis-engagement of the variable angle tips (36) with the locking assembly (34) for disassembly and/or retrieval via the suture tethers (32).

Illustrated in FIG. 3, a first embodiment of the surgical insertion device (10) of the present disclosure is provided. FIG. 3 shows the three primary regions of the surgical insertion device (10), namely, the handle portion (12), the armature portion (14), and the tip portion (16).

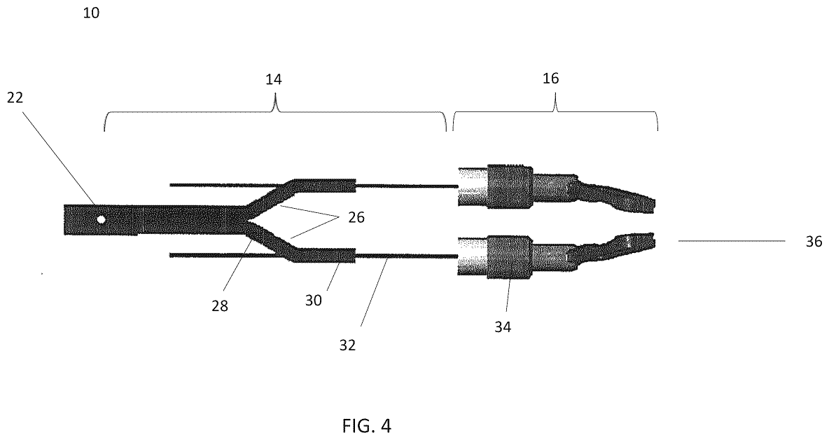

Illustrated in FIG. 4, a detail top view of the surgical insertion device (10) of FIG. 3 is provided. FIG. 4 shows the distal end (30) of each arm (26) of the armature portion (14) aligned for removable attachment to each locking assembly (34) of the tip portion (16) through tensioning the at least one suture tether (32). Alternatively, the distal end (30) of each arm (26) of the armature portion (14) may be detached from each locking assembly (34) of the tip portion (16) through the at least one suture tether (32) into an unlocked position for disassembly of the surgical insertion device (10).

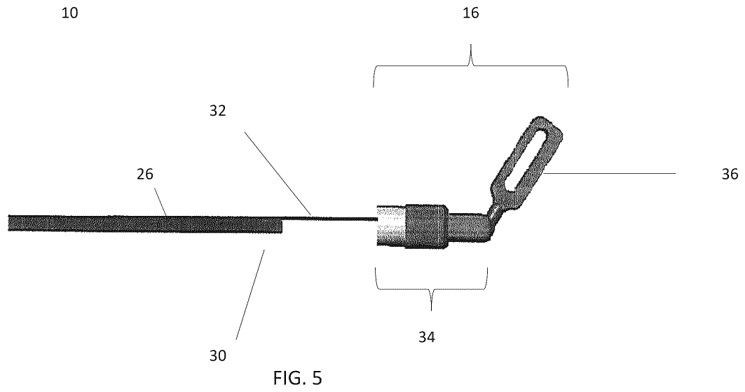

Illustrated in FIG. 5, a detail right side view of the surgical insertion device (10) of FIG. 3 is provided. FIG. 5 shows the distal end (30) of the arm (26) of the armature portion (14) aligned for removable attachment to the locking assembly (34) of the tip portion (16) through tensioning the suture tether (32). Alternatively, the distal end (30) of the arm (26) of the armature portion (14) may be detached from the locking assembly (34) of the tip portion (16) through the suture tether (32) into the unlocked position for disassembly of the surgical insertion device (10). In this nonlimiting example, the variable angle tip (36) is orientated approximately 45° to the locking assembly (34).

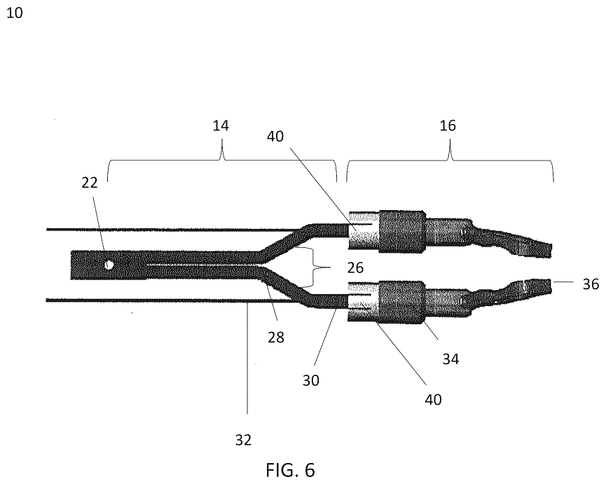

Illustrated in FIG. 6, a detail top view of the surgical insertion device (10) of FIG. 3 is provided. FIG. 6 shows the distal end (30) of each arm (26) of the armature portion (14) removably attached to each locking assembly (34) of the tip portion (16) after tensioning the at least one suture tether (32). The distal end (30) of each arm (26) of the armature portion (14) is shown in a locked position relative to each locking assembly (34) of the tip portion (16).

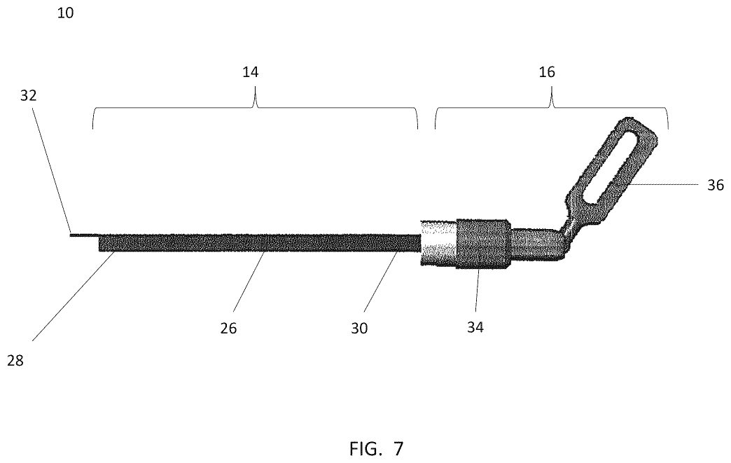

Illustrated in FIG. 7, a detail right side view of the surgical insertion device (10) of FIG. 3 is provided. FIG. 7 shows the distal end (30) of the arm (26) of the armature portion (14) removably attached to the locking assembly (34) of the tip portion (16) after tensioning the suture tether (32). The distal end (30) of the arm (26) of the armature portion (14) is shown in the locked position relative to the locking assembly (34) of the tip portion (16).

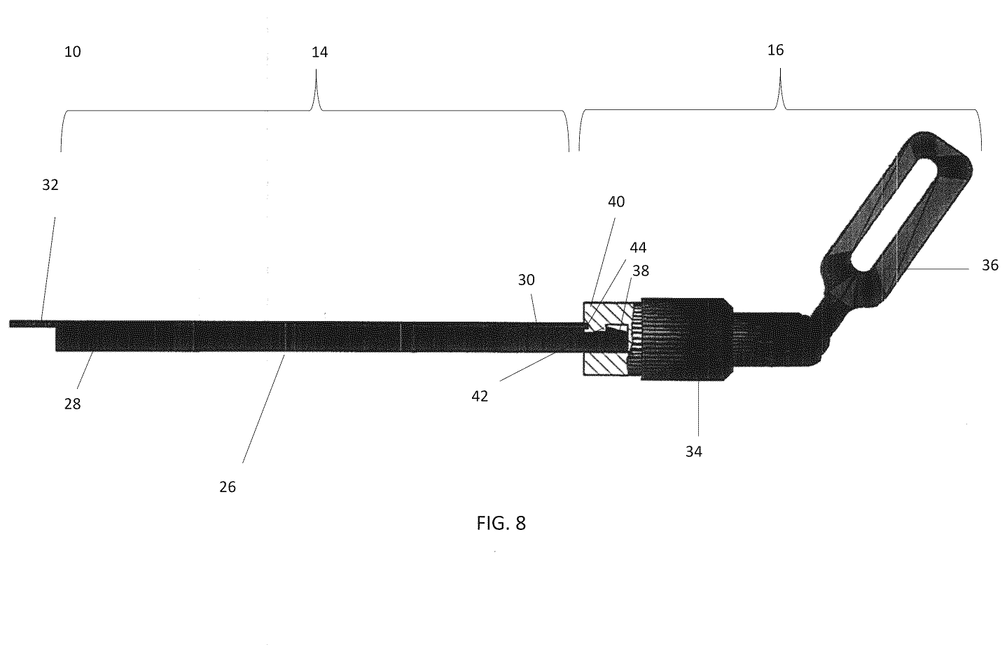

Illustrated in FIG. 8, a detail right side cross-sectional view of the surgical insertion device of FIG. 3 is provided. FIG. 8 shows the distal end (30) of the arm (26) of the armature portion (14) removably attached to the locking assembly (34) of the tip portion (16) after tensioning the suture tether (32). The distal end (30) of the arm (26) of the armature portion (14) is shown in the locked position relative to the locking assembly (34) of the tip portion (16). In particular, the locking assembly (34) may comprise a ramp (38), a flexible tab (40), a catch (42), and a release pin (44). During attachment of the distal end (30) of the arm (26) to the locking assembly (34), tensioning of the suture tether (32) is designed to cause the flexible tab (40) to move upwards, slide over the ramp (38), and move downwards against the catch (42). The flexible tab (40) against the catch (42) are configured to attach the distal end (30) of the arm (26) to the locking assembly (34) in the locked position. The flexible tab (40) against the catch (42) may also provide an audible “click” sound when fully seated to confirm that the locking assembly (34) and the distal end (30) of the arm (26) of the armature portion (14) are in the locked position.

Shown in FIG. 8, the surgical insertion device (10) may also be configured for disassembly. In particular, the distal end (30) of the arm (26) may be detached from the locking assembly (34) by using the release pin (44) to move the flexible tab (40) upwards to slide over the ramp (38). In some instances, the release pin (44) may comprise a rigid linear cylindrical pin designed to be advanced along a parallel axis to the suture tether (32) and force the flexible tab (40) upwards. After the flexible tab (40) is moved upwards, a continued translation of the release pin (44) may be configured to dis-associate the catch (42) from the flexible tab (40) to detach the distal end (30) of the arm (26) from the locking assembly (34). In such instances, the handle portion (12) and the armature portion (14) of the surgical insertion device (10) may be removed while leaving the dis-assembled tip portion (16) within the surgical site. The dis-assembled tip portion (16) may then be subsequently removed via the suture tether (32). This capability permits the independent removal of the handle portion (12), the armature portion (14), and the tip portion by pulling on the suture tether (32). This capability for disassembly offers convenient and safe removal of components of the surgical insertion device (10) from a restrictive surgical site while mitigating potential damage to surrounding tissue and nerves.

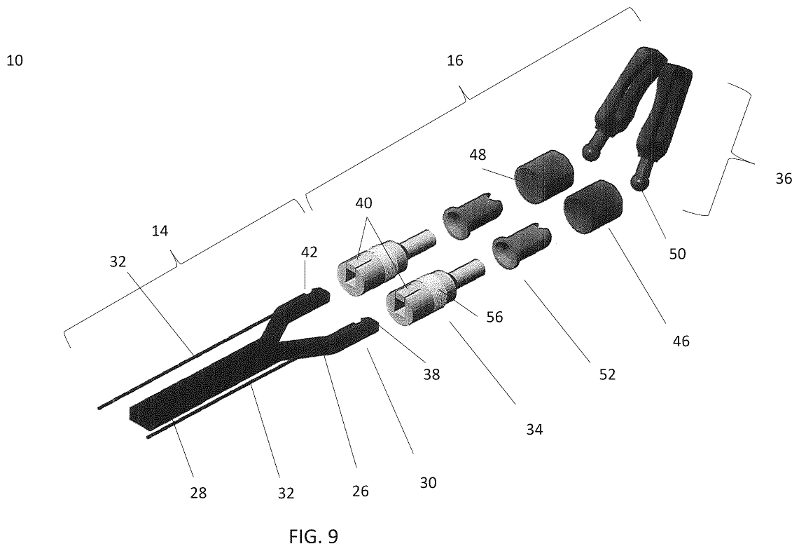

Illustrated in FIG. 9, a detail top rear isometric exploded view of the surgical insertion device (10) of FIG. 3 is provided. FIG. 9 shows components of the tip portion (16), including but not limited to, the variable angled tips (36), a collet (46) having internal threads (48), a ball (50) and socket (52) for forming a ball-and-socket joint (54), and external threads (52) on the locking assembly (34).

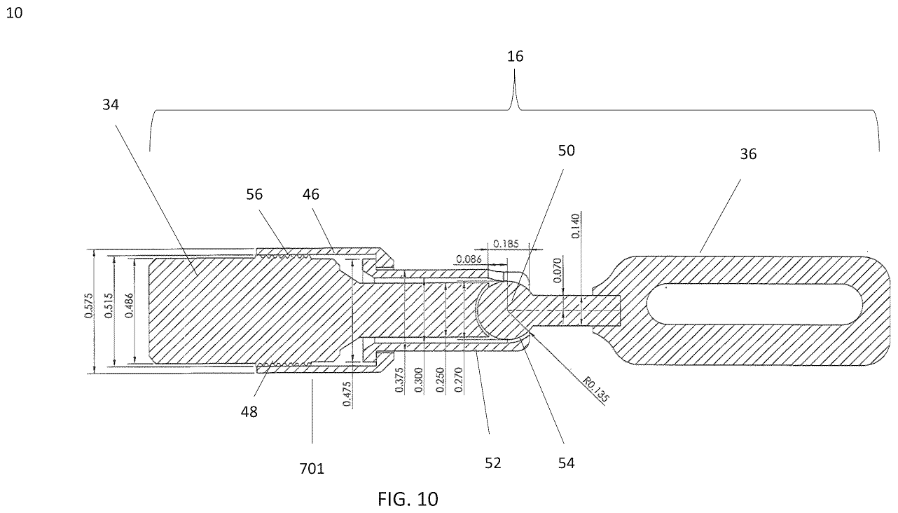

Illustrated in FIG. 10, a detail right side cross-sectional view of the surgical insertion device (10) of FIG. 3 is provided. FIG. 10 further discloses nonlimiting examples of dimension ranges. In particular, each variable angled tip (36) may comprise the ball (50) of the ball-and-socket joint (50). In some instances, the ball (50) may be manufactured integrally together with the variable angled tip (36). In other instances, the ball (50) may be manufactured separate from the variable angled tip (36) and affixed together through, e.g., adhesives, welds, or threads/screws. The ball (50) may be inserted into the socket (52) to form the ball-and-socket joint (54). The ball-and-socket joint (54) is configured to provide 360° rotation to the variable angled tip (36). The ball-and-socket joint (54) is further configured to provide the variable angled tip (36) with angular and rotational adjustment over six DOF, namely, the x axis, y axis, and z axis, with respect to the locking assembly (34). As a result, the surgical insertion device (10) of the present disclosure provides adjustable arm/tip orientation as compared to traditional surgical insertion device with fixed arm/tip orientation.

Once the variable angled tip (36) is in the desired angular and rotational orientation, the collet (46) may be utilized to lock the variable angled tip (36) in position. In particular, the collet (46) may be advanced over a distal end of the locking assembly (34) wherein the internal threads (48) of the collet (46) are mated with the external threads (56) of the locking assembly (34). When rotatably tightened, the collet (46) applies a clamping force to securely lock the ball (50) of the variable angled tip (36) in position within the ball-and-socket joint (54). The range of motion afforded by the present disclosure provides a surgeon with a variety of desired locked angular and rotational orientations of the variable angled tips (36), wherein each variable angled tip (36) of the tip portion (16) may be independently adjusted. The surgical insertion device (10) may then be used for insertion of the glenosphere during reverse shoulder replacement surgery. Indeed, providing the surgeon with the ability to intra-operatively adjust and lock the variable angled tips (36) into a precise and desired position grants access through smaller surgical sites and maneuverability around surrounding tissue for improved accuracy of glenosphere delivery.

Alternatively as opposed to using the collet (46) to lock the ball (50) of the variable angled tip (36) in position within the ball-and-socket joint (54), the ball-and-socket joint (54) may be locked in position through the introduction of a fast setting adhesive (e.g., super glue, cyanoacrylate glue). In such instances, a surgeon or clinician may apply the adhesive through low profile tubes (not shown) to deliver a controlled amount of adhesive specifically into the ball-and-socket joint (54), thereby securing the variable angled tip (36) in a desired orientation intra-operatively. It is acknowledged by the present disclosure that utilizing fast setting adhesive may limit an ability to disassemble the variable angled tip (36).

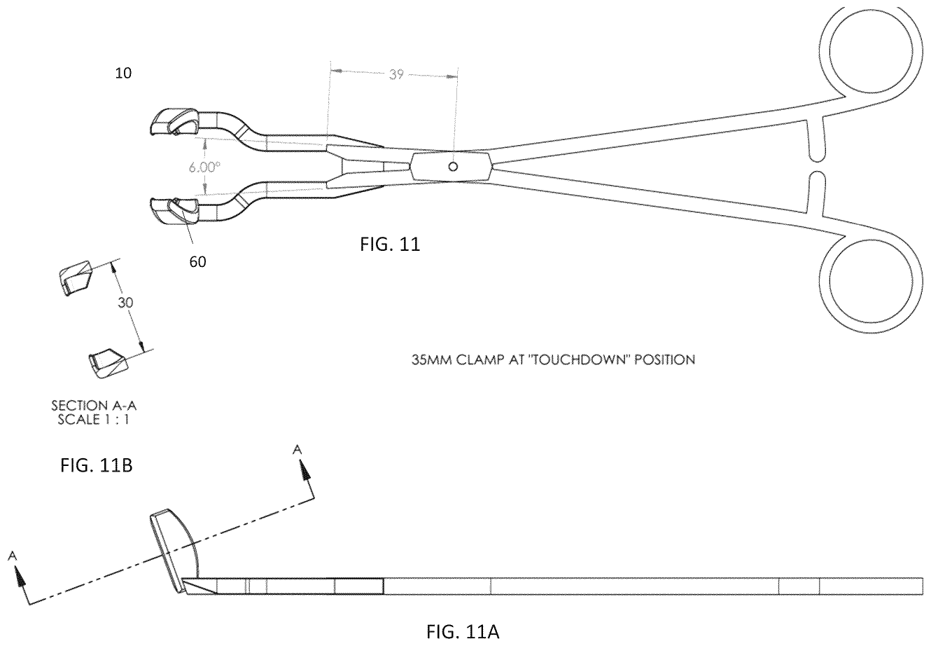

Illustrated in FIG. 11, a top view of a second embodiment of the surgical insertion device (10) of the present disclosure is provided comprising a fixed curved tip (60). In particular, the surgical insertion device (10) may comprise a fixed curved tip (60) configured to provide three point fixation on the glenosphere for better control, maneuverability, and insertion of the implant during reverse shoulder replacement surgery. The fixed curved tip (60) of the surgical insertion device (10) may also be universally applicable for use with all sizes of glenospheres. For example, the fixed curved tip (60) may be optimized to work with a full range of glenospheres, including but not limited to, 33 to 44 mm diameter ranges, and 33+ to 44+ mm diameter ranges. Because of the particular curvature design of the fixed curved tip (60), the surgical insertion device (10) is convenient to insert into the shoulder surgical site during implantation of the glenosphere during surgery in addition to convenient to extract from the shoulder surgical site after implantation. The particular curvature design of the fixed curved tip (60) further provides for eccentricity of the glenosphere relative to the baseplate. Instead of being perfectly centered, the eccentric glenosphere is offset, typically inferiorly or anteriorly, to improve insertion of the glenosphere during surgery and reduce the risk of scapular notching. The particular curvature design of the fixed curved tip (60) thus may provide improved maneuverability around hard and soft tissue during surgery while mitigating damage to surrounding tissue and nerves during trialing and implantation of the glenosphere. FIG. 11A shows a left side view of the surgical insertion device (10) of FIG. 11. FIG. 11B shows a cross-sectional view A-A of the surgical insertion device (10) of FIG. 11.

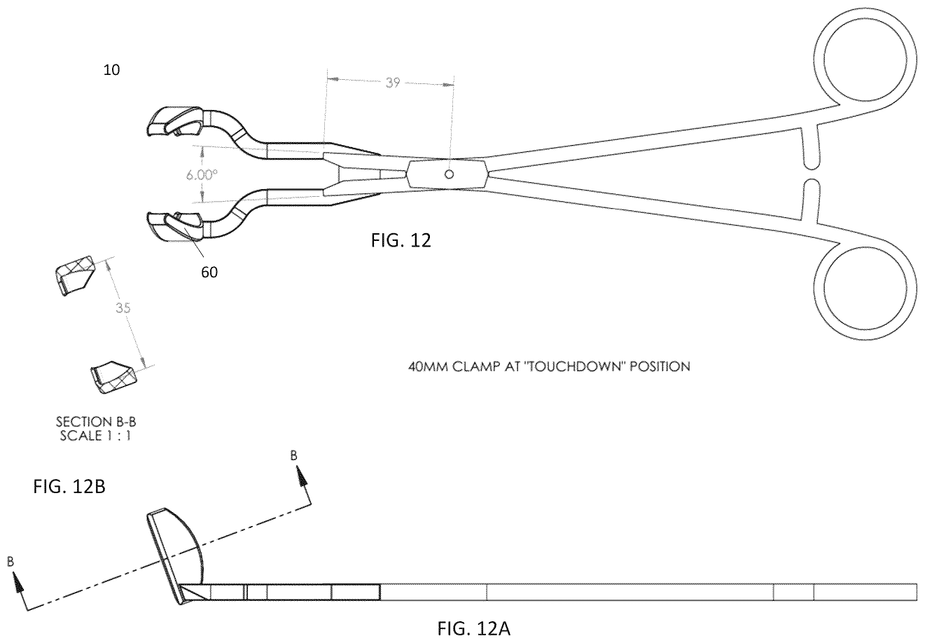

Illustrated in FIG. 12, a top view of another aspect of the surgical insertion device (10) of FIG. 11 is provided. FIG. 12A shows a left side view of the surgical insertion device of FIG. 12. FIG. 12B shows a cross-sectional view B-B of the surgical insertion device of FIG. 12.

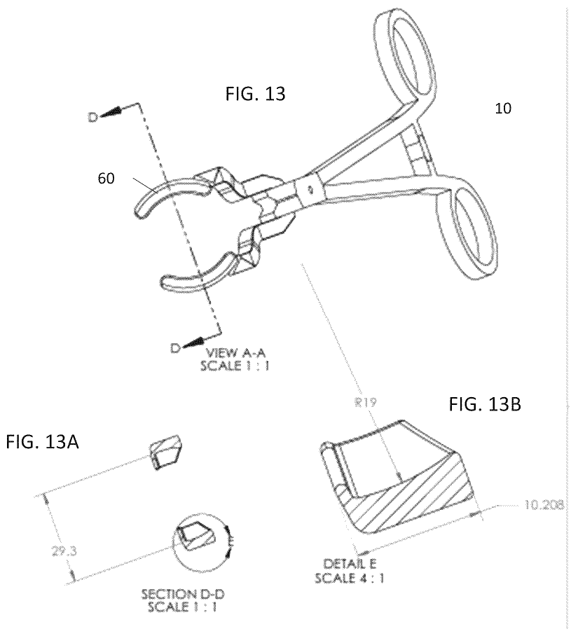

Illustrated in FIG. 13, a top front isometric view of another aspect of the surgical insertion device (10) of FIG. 11 is provided, designed for a low range of glenosphere sizes for reverse shoulder replacement surgery. FIG. 13A shows a cross-sectional view D-D of the surgical insertion device (10) of FIG. 13. FIG. 13B shows a cross-sectional enlarged view E of the surgical insertion device (10) of FIG. 13.

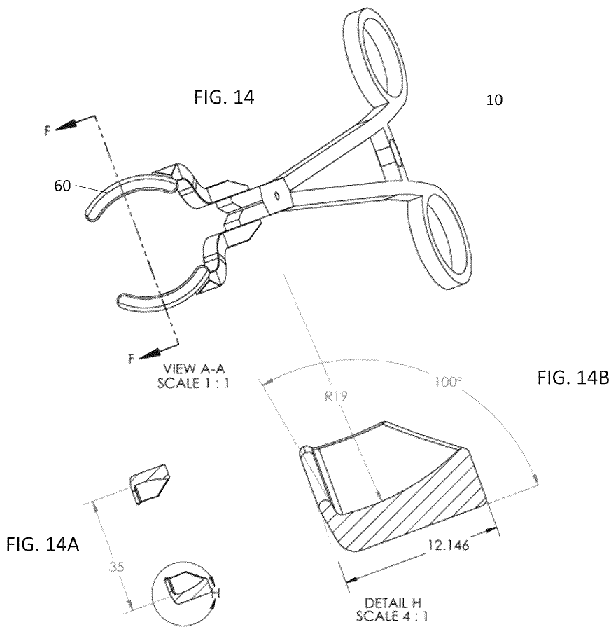

Illustrated in FIG. 14, a top front isometric view of another aspect of the surgical insertion device (10) of FIG. 11 is provided, designed for a high range of glenosphere sizes for reverse shoulder replacement surgery. FIG. 14A shows a cross-sectional view F-F of the surgical insertion device (10) of FIG. 14. FIG. 14B shows a cross-sectional enlarged view H of the surgical insertion device (10) of FIG. 14.

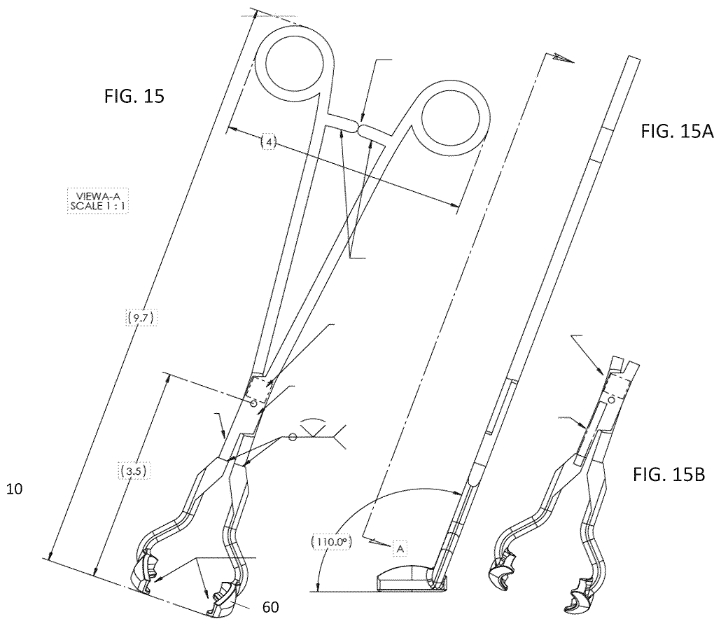

Illustrated in FIG. 15, a top view of another aspect of the surgical insertion device (10) of FIG. 11 is provided. FIG. 15A is a right side view of the surgical insertion device (10) of FIG. 15. FIG. 15B is a detail bottom view of the surgical insertion device (10) of FIG. 15.

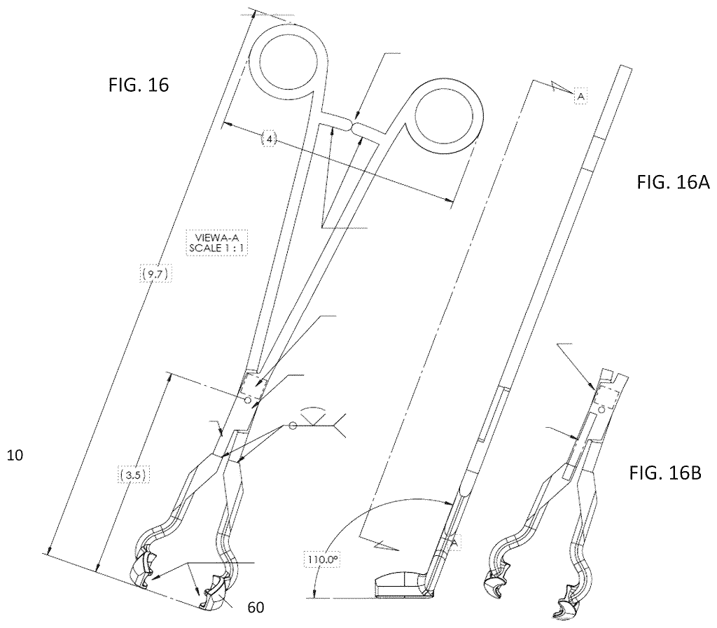

Illustrated in FIG. 16, a top view of another aspect of the surgical insertion device (10) of FIG. 11 is provided. FIG. 16A is a right side view of the surgical insertion device (10) of FIG. 16. FIG. 16B is a detail bottom view of the surgical insertion device (10) of FIG. 16.

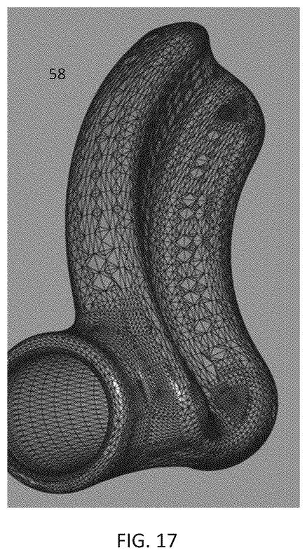

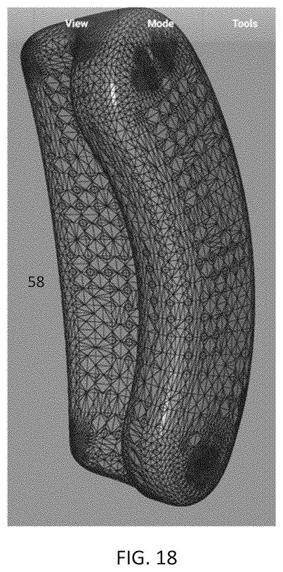

Illustrated in FIGS. 17-18, a third embodiment of the surgical insertion device (10) of the present disclosure is provided, namely, a removable cover (58) designed to protect the variable angled tip (36) and/or the fixed curved tip (60) of the surgical insertion device (10) from damage during insertion of the glenosphere during surgery. The cover (58) may be comprised of medical grade polymers, provided sterile, and intended for single use applications that are disposable and/or recyclable. The cover (58) is designed to fit snugly over the variable angled tip (36) and/or the fixed curved tip (60) of the surgical insertion device (10) to prevent the cover (58) from becoming inadvertently detached during surgical intervention. The cover (58) shown in FIGS. 17-18 is designed for side entry glenosphere insertion for reverse shoulder replacement surgery.

A fourth embodiment of the present disclosure may include a method of using the surgical insertion device (10) and cover (58) of FIGS. 3-18. The method may comprise providing the surgical insertion device (10) from a sterilization tray wherein the instrument is for reusable applications after appropriate sterilization procedures. Alternatively, the surgical insertion device (10) may be provided from a sterile package wherein the instrument is for single use applications and thereafter disposed and/or recycled. In some instances, the entire surgical insertion device (10) is for reusable applications. In other instances, the handle portion (12) and armature portion (14) is for reusable applications wherein the tip portion (16) is for single use applications.

In some instances, the method may comprise assembling the tip portion (16) and locking the tip portion (16) onto the armature portion (14) via the locking assembly (34). In particular, the distal end (30) of the arm (26) of the armature portion (14) may be removably attached to the locking assembly (34) of the tip portion (16) via tensioning the suture tether (32). In other instances, the locking assembly (34) and the distal end (30) of the arm (26) of the armature portion (14) may be removably attached using a “dove-tail” hook and latch or by leveraging two opposing snap locking “teeth.” It is contemplated that other types of removable attachment mechanisms standard in the medical industry may also be utilized with the method of the present disclosure.

In other instances, the method may comprise positioning the variable angled tip (36) in a desired angular and rotational orientation. Such adjustability of the at least one variable angle tip (36) provides access to the front of a glenosphere for better accessibility to the base plate during trialing and implantation. Once in the desired orientation, the collet (46) may be utilized to lock the variable angled tip (36) in position. In particular, the collet (46) may be rotatably tightened to apply a clamping force to securely lock the ball (50) of the variable angled tip (36) in position within the ball-and-socket joint (54). Alternatively, the ball-and-socket joint (54) may be locked in position through the introduction of a fast setting adhesive. In further instances, the method may comprise positioning the fixed curved tip (60) of the surgical insertion device (10) to maintain three point fixation on the glenosphere.

In some instances, the removable cover (58) may be optionally applied to cover the variable angled tip (36) and/or the fixed curved tip (60) of the surgical insertion device (10).

The method may further comprise utilizing the surgical insertion device (10) for side entry insertion of the glenosphere for reverse shoulder replacement surgery. In particular, the surgical insertion device (10) may be utilized for side entry at an approximate 90° angle with a patient's arm in a neutral position wherein a humeral cut is positioned approximately parallel with a glenoid. Such side entry insertion of the glenosphere using the surgical insertion device (10) of the present disclosure thus avoids obstacles such as the humerus for convenient access and implantation during surgery while mitigating potential damage to surrounding tissue and nerves. After implantation of the glenosphere, the surgical insertion device (10) may be removed from the surgical site and disposed and/or recycled or sterilized for reuse.

In other instances, after the surgical procedure is performed, the distal end (30) of the arm (26) of the armature portion (14) may be detached from the locking assembly (34) of the tip portion (16) through the suture tether (32) into the unlocked position for disassembly of the surgical insertion device (10) and the optional cover (58) thereafter disposed and/or recycled.

The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of aspects, processes, or methods of the disclosure. It is understood that any other modifications, substitutions, and/or additions can be made, which are within the intended spirit and scope of the disclosure.

What is claimed is:

1: A surgical insertion device, comprising:
a handle portion;
an armature portion; and
a tip portion;
wherein the surgical insertion device designed for side entry glenosphere insertion for reverse shoulder replacement surgery.

2: The surgical insertion device of claim 1, wherein the handle portion comprises:
a) at least one handle;
b) a pivot connection joint; and
c) at least one shank connecting the at least one handle to the pivot connection joint.

3: The surgical insertion device of claim 2, wherein the surgical insertion device is configured to provide adjustable arm/tip orientation.

4: The surgical insertion device of claim 3, wherein the armature portion comprises:
a) at least one arm;
b) each arm having a proximal end and a distal end;
c) the proximal end of each arm connected to the pivot connection joint of the handle portion; and
d) at least one suture tether configured to removably attach the distal end of each arm of the armature portion to the tip portion.

5: The surgical insertion device of claim 4, wherein the tip portion comprises:
a) at least one locking assembly;
b) at least one variable angled tip; and
c) the distal end of the arm of the armature portion configured to removably attach to the locking assembly of the tip portion through tensioning the suture tether.

6: The surgical insertion device of claim 5, wherein the at least one locking assembly comprises:
a) a ramp;
b) a flexible tab; and
c) a catch;
d) wherein the flexible tab is configured to move upwards, slide over the ramp, and move downwards against the catch to removably attach the distal end of each arm of the armature portion to the tip portion.

7: The surgical insertion device of claim 6, wherein the at least one locking assembly further comprises:
a) a release pin;
b) wherein the distal end of the arm of the armature portion is configured to be detached from the locking assembly of the tip portion using the release pin.

8: The surgical insertion device of claim 6, wherein the tip portion further comprises:
a) a collet; and
b) a ball-and-socket joint;
c) wherein the collet is configured to securely lock the variable angled tip in position within the ball-and-socket joint.

9: The surgical insertion device of claim 6, wherein the tip portion further comprises:
a) an adhesive; and
b) a ball-and-socket joint;
c) wherein the adhesive is configured to securely lock the variable angled tip in position within the ball-and-socket joint.

10: The surgical insertion device of claim 1, wherein the tip portion comprises a fixed curved tip.

11: The surgical insertion device of claim 1, further comprising a removable cover designed to protect the tip portion of the surgical insertion device.

12: A surgical insertion device, comprising:
a handle portion, comprising:
a) at least one handle;
b) a pivot connection joint; and
c) at least one shank connecting the at least one handle to the pivot connection joint;
an armature portion, comprising:
a) at least one arm;
b) each arm having a proximal end and a distal end;
c) the proximal end of each arm connected to the pivot connection joint of the handle portion; and
d) at least one suture tether configured to removably attach the distal end of each arm of the armature portion to the tip portion;
a tip portion, comprising:
a) at least one locking assembly;
b) at least one variable angled tip; and
c) the distal end of the arm of the armature portion configured to removably attach to the locking assembly of the tip portion through tensioning the suture tether;
wherein the surgical insertion device is configured to provide adjustable arm/tip orientation.

13: The surgical insertion device of claim 12, wherein the at least one locking assembly comprises:
a) a ramp;
b) a flexible tab;
c) a catch; and
d) a release pin;
e) wherein the flexible tab is configured to move upwards, slide over the ramp, and move downwards against the catch to removably attach the distal end of each arm of the armature portion to the tip portion;
a) further wherein the distal end of the arm of the armature portion is configured to be detached from the locking assembly of the tip portion using the release pin.

14: The surgical insertion device of claim 13, wherein the tip portion further comprises:
d) a collet; and
e) a ball-and-socket joint;
f) wherein the collet is configured to securely lock the variable angled tip in position within the ball-and-socket joint.

15: The surgical insertion device of claim 14, further comprising a removable cover designed to protect the variable angled tip of the surgical insertion device.

16: The surgical insertion device of claim 14, wherein the surgical insertion device is designed for side entry glenosphere insertion for reverse shoulder replacement surgery.

17: The surgical insertion device of claim 14, further comprising:
the handle portion and the armature portion are designed for reusable applications after sterilization; and
the tip portion is designed for single use applications that is disposable or recyclable.

18: The surgical insertion device of claim 14, wherein the at least one variable angle tip is configured to provide angular adjustment over six degrees of freedom.

19: A method of using a surgical insertion device, comprising:
providing a surgical insertion device, comprising:
a) a handle portion;
b) an armature portion;
c) a tip portion, the tip portion comprising at least one of a variable angled tip or a fixed curved tip; and
using the surgical insertion device to perform side entry glenosphere insertion for reverse shoulder replacement surgery.

20: The method of claim 19, further comprising detaching the tip portion from the armature portion for disassembly of the surgical insertion device.
